(19) United States
(12) Patent Application Publication
(10) Pub. No.: US 2019/0285648 A1
(43) Pub. Date: Sep. 19, 2019

(54) NOVEL LIVER CIRRHOSIS OR LIVER FIBROSIS MARKER

(71) Applicants: Fengmin LU, Beijing (CN); Mingjie YAO, Beijing (CN); Jingmin ZHAO, Beijing (CN)

(72) Inventors: Fengmin LU, Beijing (CN); Mingjie YAO, Beijing (CN); Jingmin ZHAO, Beijing (CN)

(21) Appl. No.: 16/328,846

(22) PCT Filed: Aug. 30, 2017
(86) PCT No.: PCT/CN2017/099714

§ 371 Date: Jun. 3, 2019

(30) Foreign Application Priority Data
Aug. 31, 2016 (CN) 201610798291.2

(51) Int. Cl. G01N 33/68

(52) U.S. Cl. CPC G01N 33/6893; G01N 33/57438; G01N 2800/085

(57) ABSTRACT

The invention relates to the field of diagnosis and treatment of cirrhosis and liver fibrosis. Specifically, the invention relates to the use of Golgi protein 73 (GP73) and a substance of detecting the level of Golgi protein 73 (GP73) for preparing the products for screening, auxiliarily diagnosing liver diseases or auxiliarily determining the prognosis of liver diseases. The invention proves that Golgi protein 73 (GP73) can be used as a novel marker of cirrhosis, and has diagnostic value especially for cirrhosis resulting from non-hepatitis B virus infection, but has no diagnostic value for primary hepatocellular carcinoma.

### CROSS REFERENCE TO RELATED APPLICATIONS

This application claims the benefit of priority from P.R. China Provisional Application Serial No. 201610798291.2, filed on Aug. 31, 2016, the entire content of each of which is incorporated herein by reference in its entirety.

### TECHNICAL FIELD

The invention belongs to the field of biomedicine, and relates to a novel use of a substance for detecting the concentration of Golgi protein 73 (GP73) and the use of Golgi protein 73 (GP73) in the preparation of products for screening and diagnosis of cirrhosis or liver fibrosis diseases.

### BACKGROUND

It has been 16 years since Kladney et al. discovered Golgi 73 (GP73) in the study of adult giant cell hepatitis in the year of 2000 (Kladney R D, Bulla G A, Guo L, et al. Gene. 2000. 249(1-2): 53-65). GP73, also named as Golgiphosphoprotein 2 (Golph2) and Golgi membraneprotein 1 (Golm1), is a transmembrane glycoprotein consisting of 402 amino acids and having a relative Mw of 73 kDa. It is usually located in the trans-Golgi complex. The gene encoding GP73 protein locates on chromosome 9 and the total length is 3090 nucleotides, with the encoding region locates on 199-1404 nt. GP73 is expressed in various tissues with varied expression level. It is highly expressed in small intestine, colon and stomach, and weakly expressed in liver, kidney, spleen, lung, uterus and testis, with the lowest expression level in heart. In liver tissue of normal human, GP73 is mainly expressed in the bile duct epithelial cells, but barely in the normal liver cells. Kladney et al. found in subsequent studies that the expression level of GP73 in tissues of patients with viral (HBV and HCV) infection and non-viral (alcoholic liver disease and autoimmune hepatitis) liver diseases (chronic hepatitis B, chronic hepatitis C, alcoholic cirrhosis and cirrhosis caused by autoimmune hepatitis) were significantly increased, which was 70 folds higher than those in normal liver tissues, however, there was no significant difference in between the liver disease groups. Further studies by Kladney et al. showed that GP73 expression was low in normal liver tissues. GP73 expression level was significantly increased in liver diseases caused by various factors (viral correlation, alcoholism or autoimmunity), while in the meantime, the expression of GP73 in bile duct epithelial cells did not change significantly, however, the expression of GP73 in damaged hepatocytes was significantly higher than that in pre-disease hepatocytes, indicating that the high expression of GP73 is a consequence of up-regulation of GP73 expression in hepatocytes, which is the pathological basis of the use of GP73 in the field of liver diseases (Kladney R D, Cui X, Bulla G A, Brunt E M, Fimmel C J. Hepatology. 2002. 35(6): 1431-40). Iftikhar et al. found that the expression of GP73 was increased in acute hepatitis (viral and autoimmune) and progressive cirrhosis (alcoholic liver disease and chronic hepatitis C). After immunosuppressive treatment of autoimmune hepatitis, the expression of GP73 was decreased to normal level, indicating that the initial high expression of GP73 in the disease could be reversed. Iftikhar believes that there could be two regulatory mechanisms present in the expression of GP73: one triggered during acute hepatocellular injury, the other during the progression of chronic liver disease (Iftikhar R, Kladney R D, Havlioglu N, et al. Am J Gastroenterol. 2004. 99(6): 1087-95). Block el al. made an important step in the use of GP73 in the clinical field of hepatocellular carcinoma in 2005. When they studied the relationship between the expressions of GP73 in serum of woodchuck, they found that woodchucks suffered from hepatocellular carcinoma have dramatically higher concentrations of GP73, as compared with woodchucks without a diagnosis of HCC. The same conclusion was reached when they studied the expression of GP73 in human hepatocellular carcinoma, in which the sensitivity and specificity of GP73 in the diagnosis of hepatocellular carcinoma were 69% and 75%, suggesting that the level of GP73 in serum of patients with hepatocellular carcinoma was significantly increased, and GP73 might be better than AFP in the diagnosis of early hepatocellular carcinoma (Block T M, Comunale M A, Lowman M, et al. Proc Natl Acad Sci USA. 2005. 102(3): 779-84). Cassandra therefore suggested that GP73 might be a better serum marker for the diagnosis of hepatocellular carcinoma, especially early hepatocellular carcinoma.

Marrero (Marrero J A, Romano P R, Nikolaeva O, et al. J Hepatol. 2005. 43(6): 1007-12) and Hu (Hu J S, Wu D W, Liang S, Miao X Y. Med Oncol. 2010. 27(2): 339-45) et al. studied GP73 in human hepatocellular carcinoma earlier. They believed that GP73 had significantly higher level in hepatocellular carcinoma than that in patients with hepatitis and cirrhosis, and it was better than AFP in the diagnosis of hepatocellular carcinoma. Yilei Mao et al. (Mao Y, Yang H, Xu H, et al. Gut. 2010. 59(12): 1687-93) had in-depth clinical study on GP73, and their 4217 large-sample clinical trial showed that: when GP73 with relative unit 8.5 was set as the critical value for hepatocellular carcinoma diagnosis, the diagnostic sensitivity and specificity were 74.6% and 97.4% respectively, which were higher than that in AFP detection. Combination diagnosis of GP73 and AFP showed a better effect. In the meantime, the study showed that although GP73 level was elevated with different degrees in liver benign diseases (such as hepatitis, cirrhosis) and other malignant tumors outside the liver, it was particularly prominent in the hepatocellular carcinoma. The research results of Mao et al. greatly promoted the use of GP73 in the field of hepatocellular carcinoma diagnosis. The advantage of GP73 over AFP is that it improves the detection of some AFP-negative hepatocellular carcinoma patients.

The role of GP73 in predicting the prognosis of hepatocellular carcinoma is also worth discussing. The results of Sun et al. (Sun Y, Yang H, Mao Y, et al. J Gastroenterol Hepatol. 2011. 26(7): 1207-12) showed that the serum GP73 concentration was not related to age, sex, number of tumors, liver function, size of tumors or portal vein invasion of patients, but the GP73 concentration in tumor tissue was closely related to the diameter of tumors, portal vein invasion and degree of tumor differentiation, suggesting that the concentration of GP73 in tissues is related to the malignant degree of tumors, but it fails to reveal the relationship between GP73 and patient survival.

The role of GP73 in evaluating the efficacy of cancer treatment also requires our attention. Harm et al. (Hann H W, Wang M, Hafner J, et al. Cancer Biomark. 2010. 7(6): 269-73) believe that patients with decreased GP73 concentration after first treatment can achieve nearly 6 years of tumor-free survival, while patients with increased GP73 concentration after first treatment are more likely to have recurrence within 5 years. Mao et al. also investigated the relationship between serum expression level of GP73 and tumor recurrence after hepatocellular carcinoma surgery. The results showed that the postoperative GP73 concentration decreased progressively, peaked at the 14^{th} day after surgery, and the GP73 concentration increased significantly again when the disease relapsed. The above studies suggest that GP73 may play a role in postoperative follow-up and monitoring of disease recurrence in patients with hepatocellular carcinoma.

At present, cumulative literatures have shown that GP73 can be used as a serum marker for the diagnosis of hepatocellular carcinoma, and combination detection of GP73 and AFP can improve the sensitivity and specificity of diagnosis. Previous studies have shown that GP73 may play a role in the monitoring of hepatocellular carcinoma after operation. However, in fact, there are different opinions on the role of GP73 in hepatocellular carcinoma. Morota (Morota K, Nakagawa M, Sekiya R, et al. Clin Chem Lab Med. 2011. 49(4): 711-8) and Gu (Morota K, Nakagawa M, Sekiya R, et al. Clin Chem Lab Med. 2011. 49(4): 711-8) et al. believe that GP73 can be used as a diagnostic marker for liver diseases, and its concentration in cirrhosis and hepatocellular carcinoma patients is significantly higher than that of hepatitis patients and healthy subjects, but it is not recommended as a diagnostic marker for hepatocellular carcinoma alone. Yamamoto et al. (Yamamoto K, Imamura H, Matsuyama Y, et al. J Gastroenterol. 2010. 45(12): 1272-82) showed that GP73 concentration did not change significantly before and after surgery, so it is not recommended as a clinical indicator for post-operative detection. Meanwhile, the results published previously are mostly from retrospective clinical studies. Therefore, there is still a need for higher-level evidence obtained from large-scale, prospective and multi-center cooperation to support whether GP73 really has diagnostic value for hepatocellular carcinoma or not. In addition, although the expression of GP73 is elevated in some liver diseases such as acute hepatitis (viral and autoimmune) and progressive cirrhosis (alcoholic liver disease and chronic hepatitis C), there is no statistically significant difference between the two groups. Whether GP73 can be used as a diagnostic marker for pre-hepatocellular carcinoma conditions remains to be revealed.

### SUMMARY OF THE INVENTION

The purpose of the present invention is to provide a new use of a substance for detecting the level of Golgi protein 73.

The present invention provided a new use of a substance of detecting the level of Golgi protein 73 for preparing the products for screening, auxiliarily diagnosing liver diseases or auxiliarily determining the prognosis of liver diseases.

In the present invention, Golgi protein 73 (GP73) can be used as a marker for screening, discriminating or diagnosing liver fibrosis and cirrhosis (non-hepatitis B virus infection) from healthy persons and patients with liver fibrosis and cirrhosis, and through detecting GP73 protein concentration in the screening sample in order to screen and diagnose the different degrees of liver fibrosis or cirrhosis (mainly non-hepatitis B virus infection), the results are accurate and easy to operate. Meanwhile, according to the understanding of the prior art, markers are generally used for the diagnosis, treatment and prognosis of diseases; the change and good discrimination of GP73 in different disease degree groups also fully demonstrate that those skilled in the art can use GP73 as a marker for adjuvant treatment or prognosis judgment of liver diseases (e.g., non-hepatitis B virus infected liver diseases) based on the experimental results of the present invention.

In the use described above, the substance of detecting the Golgi protein 73 level comprises Golgi protein 73 and/or Golgi protein 73 antibody;

The Golgi protein 73 is human Golgi protein 73, and the antibody against Golgi protein 73 is the antibody against human Golgi protein 73;

The human Golgi protein 73 antibody is a monoclonal antibody, a polyclonal antibody or a genetically engineered human Golgi protein 73 antibody.

In any one of the use described above, the substance of detecting Golgi protein 73 level further comprises a reagent and a required instrument for detecting the Golgi protein 73 level besides Golgi protein 73 and/or the Golgi protein 73 antibody.

Use of recombinant vector, expression cassette, transgenic cell line or recombinant strain containing Golgi protein 73 encoding gene as a biomaterial of preparing Golgi protein 73 for preparing the products for screening, auxiliarily diagnosing liver diseases or auxiliarily determining the prognosis of liver diseases also falls within the scope of protection of the present invention.

Use of recombinant vector, expression cassette, transgenic cell line, recombinant strain containing Golgi protein 73 antibody encoding gene or immunogen or antigen of Golgi protein 73 antibody as a biomaterial of preparing Golgi protein 73 antibody for preparing the products for screening, auxiliarily diagnosing liver diseases or auxiliarily determining the prognosis of liver diseases also falls within the scope of protection of the present invention.

In any one of the use described above, the disease is cirrhosis or liver fibrosis, and preferably the cirrhosis or liver fibrosis is cirrhosis or the liver fibrosis resulting from non-hepatitis B virus infection.

In any one of the use described above, subject in the screening or diagnosing by the product is a patient with cirrhosis, liver fibrosis or a healthy person.

In any one of the use described above, the product is tested in serum, plasma or interstitial fluid, and preferably human serum, human plasma or human interstitial fluid.

In any of the use described above, the product is a kit.

The present invention also provides a kit for screening, auxiliarily diagnosing liver diseases or auxiliarily determining the prognosis of liver diseases, and the kit comprises a substance for detecting Golgi protein 73 level.

In the kit described above, the substance for detecting the Golgi protein 73 level comprises Golgi protein 73 and/or Golgi protein 73 antibody.

In any of the kits described above, the disease is cirrhosis or liver fibrosis, and preferably the cirrhosis or liver fibrosis is cirrhosis or liver fibrosis of non-hepatitis B virus infection.

In any of the kits described above, detection methods by the kit include enzyme-linked immunosorbent assay, chemiluminescence, electrochemiluminescence, enzymatic chemiluminescence, time-resolved fluorescence, or upconversion luminescence.

### Beneficial Effect of the Invention

The beneficial effect of the invention is that the GP73 has a relatively modest diagnostic value for hepatocellular carcinoma, and cannot distinguish the patients with hepatocellular carcinoma from those with hepatitis and cirrhosis, and its discrimination ability is not as good as that of the AFP. On the contrary, the sensitivity of GP73 in diagnosing cirrhosis is higher than that of serum AFP, and its ability in distinguishing patients with hepatitis or cirrhosis is stronger.

### BRIEF DESCRIPTION OF THE DRAWINGS

FIG. 1 shows the ROC curve of the diagnostic value of GP73 for hepatocellular carcinoma. (A) GP73 concentrations were measured in non-cirrhosis chronic liver disease group, cirrhosis group, hepatocellular carcinoma (HCC) group, non-cirrhosis HCC group and HCC group transformed from cirrhosis. (B) The sensitivity in distinguishing HCC from non-cirrhosis chronic liver disease. (C) The sensitivity in distinguishing HCC from cirrhosis. (D)-(E) Immunohistochemical images of liver in different degrees of disease.

FIG. 2 shows the ROC curve of the diagnostic value of GP73 for cirrhosis. (A) The diagnostic value of GP73 in recruited patients with cirrhosis. (B) ROC curves of different markers in chronic hepatitis C (CHC). (C) ROC curves of different markers in chronic alcoholic liver disease (ALD). (D) The diagnostic value of GP73 for cirrhosis in all other populations (autoimmune liver disease, nonalcoholic fatty liver disease and primary biliary cirrhosis, etc.).

FIG. 3 shows a comparison of the diagnostic value of serum GP73 for liver fibrosis. (A) The GP73 concentrations in samples were measured in different degrees of liver fibrosis (F=0, 1, 2, 3 and 4). (B)-(D) ROC curves for different degrees of liver fibrosis. (E) Immunohistochemical images of liver in different degrees of fibrosis.

### DETAILED DESCRIPTION

The following makes further explanations to the present invention by specific implements and experimental data. Although the terminologies hereinafter are used for the purpose of clearness, they are not intended to define or limit the scope of the present disclosure.

As used in the text, the term “sensitivity” is also called “true-positive rate”, which refers to the percentage of patients who are actually ill and are correctly judged as ill according to the testing criteria. The greater the sensitivity is the better. The ideal sensitivity is 100%.

As used in the text, the term “specificity” is also called “true-negative rate”, which refers to the percentage of patients who are actually disease-free and are correctly judged as disease-free according to the testing criteria. The greater the specificity is the better. The ideal sensitivity is 100%.

Youden index is also called correct diagnostic index. Its value ranges between 0˜1, and the closer it is to 1, the better the diagnostic accuracy. In the method of the invention, the diagnostic criterion is set at the maximum of the correct diagnostic index.

The receiver (relative) operating characteristic (ROC) reflects the balance between sensitivity and specificity. The area under the ROC curve is an important test accuracy indicator. Calculate the area under the ROC line of each test (AUCROC) for comparison. The larger the area, the greater the diagnostic value of the test.

Unless otherwise specified, the experimental methods in the following Examples are conventional methods.

Unless otherwise specified, the materials, reagents, etc. used in the following Examples can be purchased commercially.

### EXAMPLES

### Example 1. Comparison of Diagnostic Values of Serum GP73, AFP, AFP-L3 and AFP-L3 in Primary Hepatocellular Carcinoma

1) Baseline Data of Subjects and Samples

Blood samples were obtained from: 805 patients with hepatitis; 2015 patients with liver cirrhosis; 1102 patients with liver cancer, a total of 3922 patients. See Table 1 for patient's specific information.

The samples were all serum of clinically confirmed patients. The three clinical samples were provided by the People's Liberation Army No. 302 Hospital. The patients were treated in the People's Liberation Army No. 302 Hospital from December 2013 to December 2014.

2) Statistics

SPSS 21.0 (SPSS, Chicago, Ill., USA) statistical software was used for statistical analysis. Descriptive methods of continuous variables are selected according to their distribution characteristics. Quantitative data approximates the normal distribution is expressed by x̅±S. The distribution characteristics of quantitative data with skewed distribution are described by median (M) and interquartile range. χ2 test was used for comparison among qualitative data sets, Mann-WhitnyU test was used for comparison between two independent samples, and Kruskal Wallis test was used among multiple sets of samples. The receiver operating characteristic curve (ROC curve) was established, and the value of different indicators for the diagnosis of primary hepatocellular carcinoma was compared according to the area under the curve, taking α=0.05 as testing standard.

3) Results

The commercial ELISA kit of Beijing Rejing Biotechnology Co. Ltd. was used to detect the level of GP73 in serum samples according to the specific experimental procedures of the kit instruction. The results showed that the level of GP73 in chronic hepatitis, cirrhosis and hepatocellular carcinoma were 45.20±0.78 ng/mL, 150.32±1.78 ng/mL and 121.32±2.47 ng/mL respectively, as shown in Table 1, while the level of alpha-fetoprotein AFP3 in chronic hepatitis, cirrhosis and hepatocellular carcinoma were 3.12±0.23 ng/mL, 33.51±4.64 ng/mL and 567.36±40.71 ng/mL respectively.

TABLE 1
Testing results of the subjects

| | | | Hepatocellular | |
|---|---|---|---|---|
| | Hepatitis | Cirrhosis | carcinoma | |
| | (n = 805) | (n = 2015) | (n = 1102) | |
| Variate | mean ± SEM | mean ± SEM | mean ± SEM | P |
| Age | 44.68 ± 0.39 | 50.58 ± 0.25 | 52.09 ± 0.31 | 0.000 |
| BMI (kg/m²⁾ | 24.12 ± 0.12 | 24.14 ± 0.09 | 24.00 ± 0.10 | 0.785 |
| GP73 (ng/mL) | 45.20 ± 4.78 | 150.32 ± 1.78 | 121.32 ± 2.47 | 0.000 |
| AFP3 (ng/mL) | 3.12 ± 0.23 | 33.51 ± 4.64 | 567.36 ± 40.71 | 0.000 |
| ALT (U/L) | 46.47 ± 0.14 | 93.64 ± 5.31 | 72.23 ± 4.68 | 0.000 |
| AST (U/L) | 35.27 ± 1.61 | 87.47 ± 3.61 | 79.36 ± 4.25 | 0.000 |
| PLT (10\hat{ }9/L) | 187.71 ± 12.20 | 104.14 ± 1.47 | 144.85 ± 2.75 | 0.000 |
| APRI | 0.56 ± 0.04 | 2.71 ± 0.14 | 2.05 ± 0.16 | 0.000 |
| FIB-4 | 1.57 ± 0.07 | 6.92 ± 0.21 | 4.61 ± 0.17 | 0.000 |

Note:
ALT, alanine aminotransferase;
AST, glutamic oxaloacetic aminotransferase;
PLT, platelet;
APRI, a scoring method, the ratio index of aspartate aminotransferase (AST) and platelet (PLT);
FIB-4, the evaluation index of liver disease.

4) Comparison of the Diagnostic Values of Serum GP73, AFP, AFP-L3 and AFP-L3 in Primary Hepatocellular Carcinoma

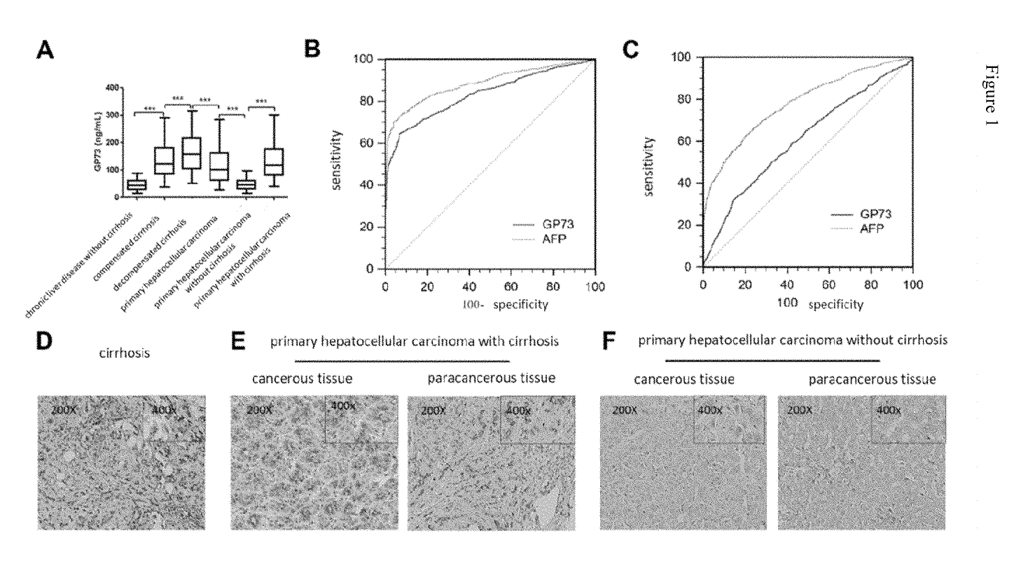

Using the pathological diagnosis as the gold standard, the ROC curve was used to calculate the sensitivity, specificity, positive predictive value, negative predictive value, false positive rate and Youden index of serum GP73, AFP, AFP-L3 and AFP-L3, and calculated the best diagnostic value. The results showed that the area under the ROC curves of serum Golgi protein 73 (GP73), alpha-fetoprotein (AFP) and alpha-fetoprotein heterogenous (AFP-L3) were 0.527 (95% CI, 0.503-0.552), 0.826 (95% CI, 0.808-0.843 P<0.001) and 0.824 (95% CI, 0.806-0.841 P<0.001) respectively, as shown in FIG. 1 and Table 2.

TABLE 2
The diagnostic values of GP73, AFP, AFP-L3 (%) and AFP-L3 in
hepatocellular carcinoma

| | | | | Area | |
|---|---|---|---|---|---|
| | | | | under | |
| | | | Youden | the curve | |
| Variate (%) | Sensitivity | Specificity | index | [symbol] | 95% CI |
| AFP | 68.56% | 82.45% | 0.510 | 0.826 | 0.808-0.843 |
| AFP-L3 | 67.60% | 82.65% | 0.503 | 0.824 | 0.806-0.841 |
| AFP-L3 | 54.58% | 88.02% | 0.462 | 0.677 | 0.653-0.701 |
| GP73 | 85.25% | 21.89% | 0.071 | 0.527 | 0.503-0.552 |

[symbol]

indicates data missing or illegible when filed

### Example 2. The Distribution Characteristics of Serum GF73, AFP and AFP-L3(%) in Patients with Hepatitis, Cirrhosis and Hepatocellular Carcinoma

The subjects and statistical methods were the same as in Example 1. In the experiment, the changes of AFP, AFP-L3(%) and GP73 in patients with hepatitis, cirrhosis and hepatocellular carcinoma were described by interquartile range. The results showed that the trend of GP73 change in hepatitis, cirrhosis and hepatocellular carcinoma was obvious, but it could not distinguish patients with hepatocellular carcinoma from those with hepatitis and cirrhosis. AFP and AFP-L3(%) showed significant change in patients with liver cancer only, and could distinguish patients with hepatocellular carcinoma from those with hepatitis and cirrhosis, as shown in Table 3.

TABLE 3
Interquartile ranges of GP73, AFP and AFP-L3 (%) in
patients with hepatitis, cirrhosis and hepatocellular carcinoma

| | | Interquartile range | | | | | | |
|---|---|---|---|---|---|---|---|---|
| Variate | Disease | 5 | 10 | 25 | 50 | 75 | 90 | 93 |
| GP73 | Hepatitis | 15.291 | 20.568 | 33.500 | 57.480 | 90.573 | 159.230 | 219.535 |
| | Cirrhosis | 26.950 | 39.260 | 72.220 | 121.600 | 179.200 | 246.400 | 277.900 |
| | Hepatocellular | 29.432 | 38.750 | 61.960 | 103.800 | 168.350 | 240.080 | 291.640 |
| | Carcinoma | | | | | | | |
| AFP | Hepatitis | 0.896 | 1.049 | 1.490 | 2.260 | 3.600 | 10.697 | 52.828 |
| | cirrhosis | 0.822 | 1.030 | 1.600 | 2.660 | 5.820 | 20.170 | 72.520 |
| | Hepatocellular | 1.740 | 2.268 | 5.145 | 40.850 | 885.850 | 1300.000 | 2245.300 |
| | Carcinoma | | | | | | | |
| AFP-L3 | Hepatitis | 0.045 | 0.053 | 0.075 | 0.114 | 0.181 | 0.690 | 5.419 |
| | Cirrhosis | 0.041 | 0.052 | 0.081 | 0.135 | 0.299 | 1.665 | 7.130 |
| | Hepatocellular | 0.087 | 0.111 | 0.256 | 3.800 | 152.600 | 1018.300 | 1100.000 |
| | carcinoma | | | | | | | |

### Example 3. Comparison of the Diagnostic Values of Serum GP73, LSM, FIB4 and APRI in Cirrhosis

The subjects and statistical methods were the same as in Example 1.

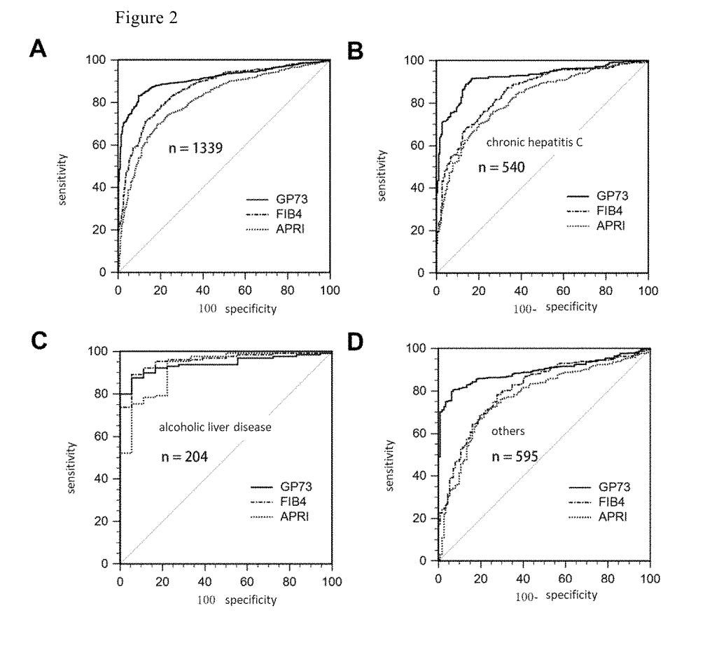

The results of this part showed that the sensitivity and specificity of GP73 in the diagnosis of cirrhosis were 83.97% and 93.54% respectively, and the area under curve was 0.927 (95% CI, 0.917-0.937) (FIG. 2). The diagnostic value of GP73 was better than that of liver stiffness measurement (LSM), FIB-4 index and platelet (PLT) ratio index APRI, as shown in Table 4, indicating that GP73 had a stronger ability to distinguish patients with cirrhosis.

TABLE 4
The diagnostic values of GP73, LSM, APRI and FIB4 in cirrhosis

| | | | | Positive | Negative | | Area | | |
|---|---|---|---|---|---|---|---|---|---|
| | cut-off | Sensitivity | Specificity | predictive | Predictive | Youden | under | | |
| Variate | value | (%) | (%) | value % | value % | index | the curve | 95% CI | P |
| GP73 | 75.16 | 83.97 | 93.54 | 97.02 | 69.99 | 0.775 | 0.927 | 0.917-0.937 | <0.0001 |
| ng/ml | | | | | | | | | |
| LSM | 11.8 | 74.19 | 86.75 | 90.63 | 66.06 | 0.609 | 0.885 | 0.866-0.901 | <0.0001 |
| kPa | | | | | | | | | |
| FIB-4 | 2.0 | 83.17 | 82.75 | 92.35 | 66.27 | 0.659 | 0.900 | 0.888-0.910 | <0.0001 |
| APRI | 0.64 | 78.92 | 81.95 | 91.63 | 60.84 | 0.609 | 0.871 | 0.858-0.883 | <0.0001 |

### Example 4. Comparison of the Diagnostic Values of Serum GP73 in Liver Fibrosis

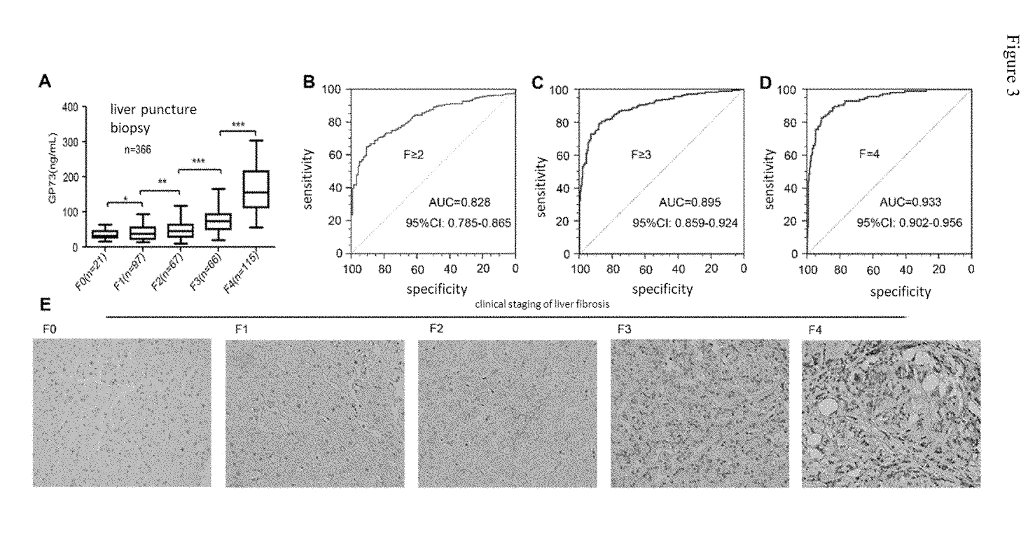

Serum GP73 could well distinguish between significant liver fibrosis (F≥2), severe liver fibrosis (F≥3) and early cirrhosis (F=4). Serum GP73 level (Means±SE) increased with the degree of liver fibrosis F0 (n=21, 5.74%): 35.18±2.95; F1 (n=97, 26.50%): 42.73±2.57; F2 (n=67, 18.31%): 50.59±3.48; F3 (n=66, 18.03%): 79.93±5.67; F4 (n=115, 31.42%): 169.26±7.31 (P<0.001). The sensitivity, specificity and AUROC of serum GP73 in the diagnosis of F≥2, F≥3 [symbol] F=4 were F≥2: 64.92%, 90.68%, 0.828 (95% CI: 0.785-0.865); F≥3: 80.66%, 86.49%, 0.895 (95% CI: 0.859-0.924); F=4: 82.61%, 91.24%, 0.933 (95% CI: 0.902-0.956), as shown in FIG. 3.

The above results indicate that GP73 can be used as a potential marker for screening and discriminating liver fibrosis and cirrhosis (non-hepatitis B virus infection) from healthy people and patients with liver fibrosis and cirrhosis, and the method of screening and diagnosing different degrees of liver fibrosis or cirrhosis (non-hepatitis B virus infection) by detecting the GP73 protein concentration in samples of the screening target is accurate and easy to operate.

The Examples of the present invention have been described above, but the present invention is not limited thereto, and those skilled in the art can understand that modifications and changes can be made within the scope of the purport of the present invention. The manner of modifications and changes should fall within the scope of protection of the present invention.

1. Use of a substance of detecting Golgi protein 73 level for preparing the products for screening, auxiliarily diagnosing liver diseases or auxiliarily determining the prognosis of liver diseases.

2. The use of claim 2, wherein the substance of detecting Golgi protein 73 level comprises Golgi protein 73 and/or Golgi protein 73 antibody.

3. The use of claim 3, wherein the substance of detecting Golgi protein 73 level further comprises a reagent and a required instrument for detecting the Golgi protein 73 level.

4. Use of recombinant vector, expression cassette, transgenic cell line or recombinant strain containing Golgi protein 73 encoding gene as a biomaterial of preparing Golgi protein 73 for preparing the products for screening, auxiliarily diagnosing liver diseases or auxiliarily determining the prognosis of liver diseases.

5. Use of recombinant vector, expression cassette, transgenic cell line, recombinant strain containing Golgi protein 73 antibody encoding gene or immunogen or antigen of Golgi protein 73 antibody as a biomaterial of preparing Golgi protein 73 antibody for preparing the products for screening, auxiliarily diagnosing liver diseases or auxiliarily determining the prognosis of liver diseases.

6. The use of any one of claims 1 to 5, wherein the disease is cirrhosis or liver fibrosis, and preferably the cirrhosis or the liver fibrosis is cirrhosis or liver fibrosis resulting from non-hepatitis B virus infection.

7. The use of any one of claims 1 to 5, wherein subject in the screening or diagnosing by the product is a patient with cirrhosis, liver fibrosis or healthy person.

8. The use of any one of claims 1 to 5, wherein the product is tested in serum, plasma or interstitial fluid, and preferably human serum, human plasma or human interstitial fluid.

9. The use of any one of claims 1 to 5, wherein the product is a kit.

10. A kit for screening, auxiliarily diagnosing liver diseases or auxiliarily determining the prognosis of liver diseases, and the kit comprises a substance for detecting Golgi protein 73 level.

11. The kit of claim 10, wherein the substance of detecting the Golgi protein 73 level comprises Golgi protein 73 and/or Golgi protein 73 antibody.

12. The kit of any one of claim 10 or 11, wherein the disease is cirrhosis or liver fibrosis, and preferably the cirrhosis or liver fibrosis is cirrhosis or liver fibrosis resulting from non-hepatitis B virus infection.

13. The kit of any one of claim 10 or 11, wherein detection methods by the kit include enzyme-linked immunosorbent assay, chemiluminescence, electrochemiluminescence, enzymatic chemiluminescence, time-resolved fluorescence, or upconversion luminescence.
